(19) United States
(12) Patent Application Publication
(10) Pub. No.: US 2021/0386943 A1
(43) Pub. Date: Dec. 16, 2021

(54) INHALER DETECTION

(71) Applicant: KINDEVA DRUG DELIVERY L.P., St. Paul, MN (US)

(72) Inventors: Peter D. Hodson, Bracknell (GB); Parfes Mohammed, Bracknell (GB); Adam J. Stuart, Loughborough (GB); Paul H.R. Jolly, Bracknell (GB)

(21) Appl. No.: 17/419,436

(22) PCT Filed: Dec. 27, 2019
(86) PCT No.: PCT/IB2019/061415

§ 371 Date: Jun. 29, 2021

(30) Foreign Application Priority Data
Dec. 31, 2018 (GB) 1821331.4

(51) Int. Cl. A61M 15/00

(52) U.S. Cl. CPC A61M 15/0006; A61M 15/0021; A61M 2205/103; A61M 2205/8206; A61M 2205/3306; A61M 15/0091

(57) ABSTRACT

An inhaler having an air inlet, a medicament source, and an outlet for delivering an air and medicament admixture to a user, the inhaler including a primary power supply for powering the controller and a sensor for detecting a recent shake event, wherein, the sensor is independent of the primary power source and, in use, the sensor provides the controller with a signal indicative of any recent shake event upon the powering up of the controller prior to use of the inhaler.

### FIELD

The present application relates to medicinal inhalers, and in particular to an inhaler capable of detecting whether sufficient mixing of the medicament has been achieved prior to dispensing, and a method of achieving the same.

### BACKGROUND

Delivery of aerosolized medicament to the respiratory tract for the treatment of respiratory and other diseases is conventionally done using inhalers of either the pressurised metered dose inhaler (pMDI), the dry powder inhaler (DPI) or the nebulizer type. pMDI inhalers in particular have become an industry standard, and are familiar to many patients who suffer from either asthma or from chronic obstructive pulmonary disease (COPD). Conventional pMDI devices comprise an aluminum canister, sealed with a metering valve, which contains the medicament formulation. Generally, the medicament formulation is a pressurized formulation containing either fine particles of one or more medicinal compounds suspended in a liquefied hydrofluoroalkane (HFA) propellant, or a solution of one or more medicinal compounds dissolved in a propellant/co-solvent system. Formulations incorporating one drug in solution and another one in suspension form are also known.

Dry powder inhalers are often described as “breath-actuated” because many of them utilise the energy of a patient's inhaled breath to release a dose of powdered medicament, usually admixed with a suitable carrier powder, for the patient to inhale directly. However, some DPIs are designed to dispense the dose actively by releasing a separate energy source to the powder, upon activation of a mechanism by the patient's inhalation.

In a conventional pulmonary pMDI, the sealed canister is provided to the patient in an actuator. The actuator is conventionally a generally L-shaped plastic molding comprising a generally cylindrical vertical tube that surrounds the canister plus a generally horizontal tube that forms a patient portion (e.g., a mouthpiece or nosepiece) that defines an inspiration (or inhalation) orifice. To use such an inhaler, the patient exhales, places the patient port into a body cavity (e.g., a mouth or nose) and then inhales to draw air through the inspiration orifice. The majority of such inhalers are of the pulmonary “press-and-breathe” type, where the patient must press down on the protruding end of the canister in order to operate the metering valve to release a metered dose of medicament from the canister into the inhaled air stream and thence through the mouthpiece into their lungs.

To overcome what can be quite a challenge for some patients, pMDI device designs have been created that employ automatic breath-actuated triggering, releasing a dose only in response to the patient's inhaled breath. The AUTOHALER™ metered dose inhaler, available from 3M Company, St. Paul, Minn., USA, and the EASIBREATHE™ inhaler, available from Teva Pharmaceutical Industries Ltd., Israel, are two such pMDI devices that use breath-actuation to attempt to better coordinate dose release with inhalation.

It is also known to provide inhalers which contain electronics in order to monitor use of the inhaler. For example, the frequency of use and the duration of each inhalation may be electronically monitored.

Both conventional and breath actuated inhalers typically contain inhaled medicament formulations which require shaking shortly before use. Electronic inhalers offer the possibility of monitoring whether such shaking occurs prior to delivery of a dose. However, in known electronic inhalers such monitoring is only possible after the device has been switched on (“woken up”). Unfortunately, it is possible that a patient might shake their electronic inhaler prior to switching it on to take a dose. In such a circumstance the electronics would not recognise that the inhaler has been shaken. Conversely, with some inhalers it is desirable that patients shake their inhaler before they wake it up. For example, in a mechanically breath actuated inhaler, the action of opening the mouthpiece cover wakes up the inhaler electronics and simultaneously sets the mechanical breath actuation system ready for triggering. It is therefore desirable that the inhaler is not shaken following the setting of the mechanical breath actuation system to avoid inadvertent firing of the mechanism. The user is therefore trained to shake the device prior to opening the mouthpiece. However, known inhalers are not able to monitor whether such a shake event has occurred as the electronics have not yet been awoken by the opening of the mouthpiece cover.

The object of the present disclosure is to at least mitigate some of the above problems.

### SUMMARY

According to a first aspect of the present disclosure there is provided an inhaler having an air inlet, a medicament source, and an outlet for delivering an air and medicament admixture to a user, the inhaler including

a primary power supply for powering the controller and

a sensor for detecting a recent shake event,

wherein, the sensor is independent of the primary power source and, in use, the sensor provides the controller with a signal indicative of any recent shake event upon the powering up of the controller prior to use of the inhaler.

Advantageously, the present disclosure allows the inhaler to determine whether the inhaler has been shaken within a predetermined time period prior to the powering up of the controller, that is to say whether there has been a recent shake event prior to powering up. This enables the inhaler to determine whether the medicament source is sufficiently agitated (and therefore whether the inhaler is ready for use) whilst permitting the controller to remain in a powered down state when not in use in order to preserve battery life.

In embodiments, any recent shake event is a shake event occurring in the range of up to 5 seconds, preferably up to 8 seconds and most preferably up to 10 seconds, prior to the powering up of the controller.

In embodiments, the controller interrogates the sensor upon powering up to determine the presence or absence of a signal indicative of a recent shake event.

In embodiments, the plurality of curved coverplates further includes a rear coverplate curved in both the longitudinal and transverse plane,

In embodiments, the controller indicates to the user that the inhaler must be shaken in the event of an absence of a signal indicative of a recent shake event.

In embodiments, the controller processes the signal indicative of a recent shake event to determine whether the shake event was sufficient to mix the medicament admixture for subsequent inhalation by the user.

In embodiments, the controller determines that the recent shake event was sufficient to mix the medicament admixture for subsequent inhalation by the user and advises the user accordingly.

In embodiments, the controller determines that the recent shake event was insufficient to mix the medicament admixture and the controller indicates to the user that the inhaler must be shaken prior to use.

In embodiments, the sensor is a mechanical sensor which exhibits residual movement initiated by the shake event.

In embodiments, the residual movement of the mechanical sensor is the signal indicative of any recent shake event.

In embodiments, the inhaler includes an optical sensor powered by the primary power source and the controller interrogates the mechanical sensor using the optical sensor upon powering up of the controller to determine whether any shake event was sufficient to mix the medicament admixture.

In embodiments, the mechanical sensor comprises an oscillating mass or a rotating mass.

Alternatively, the sensor is a fluid suspension containing a plurality of suspended particles which disperse during a shake event and settle out of suspension over a period of time after the shake event.

In embodiments, the extent of dispersion of the suspended particles is the signal indicative of any recent shake event.

In embodiments, wherein the inhaler includes a photoelectric sensor powered by the primary power source and the controller interrogates the fluid suspension sensor using the photoelectric sensor upon powering up of the controller to measure the opacity of the fluid suspension in order to determine whether any recent shake event was sufficient to mix the medicament admixture.

Alternatively, the sensor is an electronic charge capture circuit which captures a charge indicative of any recent shake event.

In embodiments, the circuit includes a vibration sensitive switch, a battery independent of the primary power supply, a capacitor for the storing the charge and a first resistor arranged in series, and a second resistor arranged in parallel with the capacitor.

In embodiments, the voltage across the capacitor is the signal indicative of any recent shake event.

In embodiments, the controller measures the voltage across the capacitor upon powering up of the controller in order to determine whether any recent shake event was sufficient to mix the medicament admixture.

In embodiments, the powering up of the controller is initiated by the user pressing a power button on the inhaler or opening a mouthpiece cover of the inhaler.

According to a first aspect of the present disclosure there is provided a method of detecting a recent shake event in a medicinal inhaler, including the steps of

providing an inhaler having

an air inlet, a medicament source, and an outlet for delivering an air and medicament admixture to a user, a primary power supply for powering the controller and a sensor for detecting a recent shake event, wherein, the sensor is independent of the primary power source,

the method further including the steps of

powering up the controller by switching on the inhaler,
the controller interrogating the sensor to determine the presence or absence of a signal indicative of any recent shake event,
the controller indicating to the user whether the inhaler must be shaken prior to use.

### BRIEF DESCRIPTION OF THE DRAWINGS

The embodiments of the present disclosure will now be described, by way of example only, and with reference to the following drawings, in which:

FIG. 1A is a side view of an inhaler according to the present disclosure shown in its closed state;

FIG. 1B is a side view of an inhaler according to the present disclosure shown in its primed state;

FIG. 2 is a schematic representation of a first embodiment of a recent shake detection sensor for use in the inhaler of FIG. 1;

FIG. 3A is a schematic representation of a second embodiment of a recent shake detection sensor for use in the inhaler of FIG. 1 shown in its rest condition;

FIG. 3B is a schematic representation of a second embodiment of a recent shake detection sensor for use in the inhaler of FIG. 1 shown in its dispersed condition; and

FIG. 4 is a schematic representation of a third embodiment of a recent shake detection sensor for use in the inhaler of FIG. 1.

### DETAILED DESCRIPTION

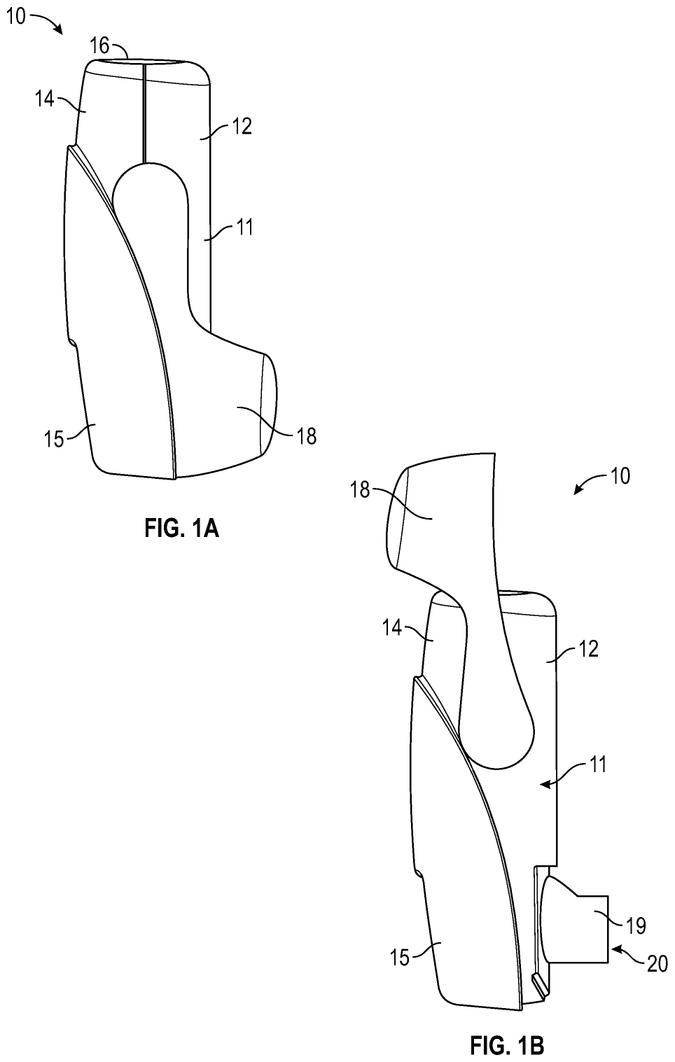

Referring initially to FIGS. 1A and 1B, an inhaler in the form of pressurized metered dose inhaler (pMDI) 10 is shown having a chassis 11 which has an upwardly extending main body 12 which houses a medicament source in the form of a canister (not shown for clarity) containing a medicament formulation.

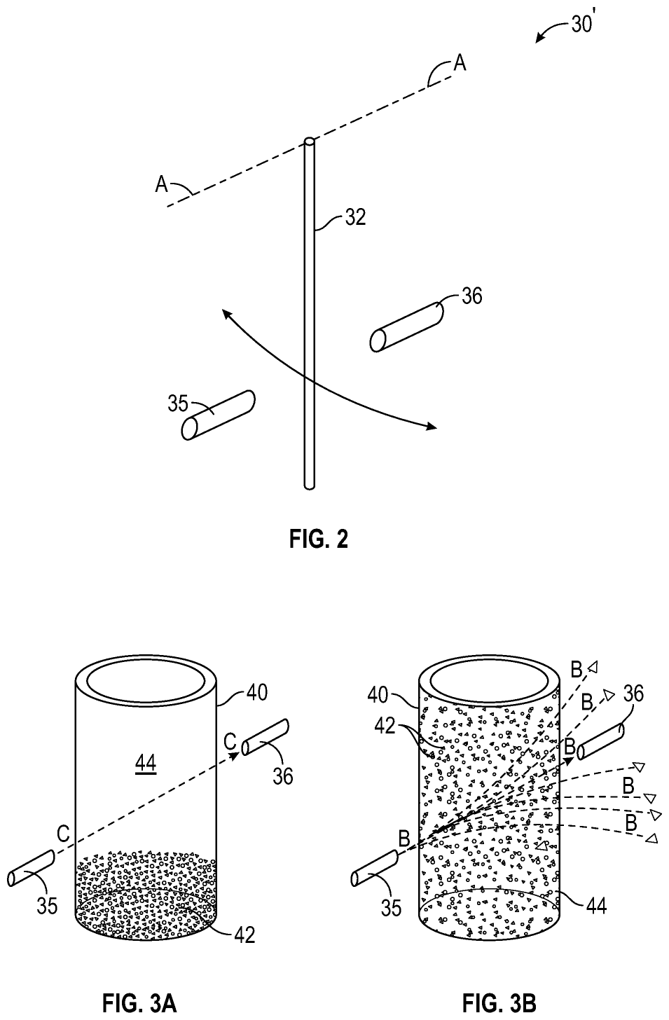

The chassis 11 has a top section 14 and a lower section 15. The top section 14 defines an air inlet 16 which permits air to enter the chassis 11. The lower section 15 has a mouthpiece cover 18 which covers a mouthpiece 19 (shown in FIG. 2B only) which defines an outlet 20 from the inhaler 10. An air flowpath (not shown for clarity) is defined between the inlet 16 and outlet 20.

The medicament is dispensed from the canister into the air flowpath upon the triggering of a breath actuation mechanism (not shown) in a known manner.

Accordingly, in use, the user moves the cover 18 from its closed position (FIG. 3A) to its primed position (see FIG. 3B) to prime the breath actuation mechanism. The user then places their mouth over the mouthpiece 19 and inhales to draw air into the main body 11 via the air inlet 16. The air is mixed with medicament from the canister, upon the triggering of the mechanical breath actuation mechanism in a known manner, to form a medicament admixture. The medicament admixture is then drawn into the mouthpiece 19 and then the mouth by the continued inhalation of the user.

The main body 12 of the inhaler houses a controller 22 which performs a number of functions including, for example, the monitoring and counting of doses, and connection to ancillary devices such as a smartphone. The controller is powered by a primary power source in the form of a battery 24.

The controller remains in a powered down state, that is to say a state in which it draws little to no power from the battery 24 until such time as the mouthpiece 18 is opened. The action of opening the mouthpiece cover 18 also sets the mechanical breath actuation system ready for triggering. It is therefore desirable that the inhaler is not shaken following the setting of the mechanical breath actuation system in case this causes inadvertent firing of the mechanism. The user is therefore trained to shake the device prior to opening the mouthpiece.

It is desirable to ensure that the user has complied with these instructions by monitoring whether the inhaler has indeed been shaken prior to the mouth piece being opened. However, this would rely on the controller remaining in a continually powered up state which would significantly negatively impact on battery life.

Accordingly, the inhaler 10 is provided with a sensor 30 (not shown in FIG. 1) for detecting a recent shake event. A shake event is defined as being recent if it occurred within a predetermined period of time prior to the inhaler being primed for use by the opening of the mouthpiece cover 18. The predetermined period of time is in the range of 5 to 10 seconds depending on the formulation being dispensed by the inhaler 10. The sensor 30 is not powered by the battery 24 and is able to operate independently of the battery 24.

Turning now to FIG. 2, a first embodiment of sensor 30 is shown as mechanical sensor 30′. The sensor 30′ has a pendulum 32 which is caused to rotate about pivot axis A by the shaking of the inhaler 10. The momentum of the pendulum 32 causes the residual movement in the pendulum to be maintained after the shaking event has ceased. The residual movement of the pendulum constitutes a signal indicative of a recent shake event.

Upon the powering up of the controller by the opening of the mouth piece cover 18, the pendulum 32 is interrogated by an optical sensor 34 powered by the battery 24 and in communication with the controller 32. The optical sensor 34 has an LED (Light Emitting Diode) 35 and a pin photodiode 36. The optical beam is directed at the pin photodiode 36 and is bisected by the path of the pendulum 32 as the pendulum 32 rotates about axis A. The controller manipulates the signal from the optical sensor 34 by software or firmware algorithm to determine whether the intensity and/or periodicity of the detected residual motion (e.g., the pattern of temporal breaks in the light beam) constitutes an adequate shake sufficiently close to the time of dose release. A time-stamped electronic record is made of the dosing event in the controller 22, along with an electronic record of the shaking.

If an adequate shake event is determined by the controller 22 to have occurred within the predetermined period of time prior to powering up, the user is informed that the inhaler 10 is ready for use. Equally, if the controller 22 determines that no adequate shake event has occurred within the predetermined period of time prior to powering up, the user is informed that the inhaler must be closed and shaken prior to use. Such information could be presented aurally or on a screen positioned on the inhaler 10.

Turning now to FIGS. 3A and 3B, a second embodiment of sensor 30 is shown as fluid suspension 30″. The sensor 30″ comprises a transparent, or substantially transparent vessel 40. The vessel 40 contains a plurality of particles 42 which are suspended in a clear fluid 44.

In FIG. 3A, the particles 42 have fallen out of suspension within the fluid 44 and are shown to have agglomerated at the base of the vessel 40. In FIG. 3B, by contrast, the particles are fully dispersed throughout the fluid 42 having been agitated by a recent shake event. The extent of dispersion of the suspended particles constitutes a signal indicative of any recent shake event.

Upon the powering up of the controller by the opening of the mouth piece, the vessel is interrogated by an optical sensor 34 powered by the battery 24 and in communication with the controller 22. The sensor 34 has an LED (Light Emitting Diode) 35 and a pin photodiode 36. The optical beam is directed at the pin photodiode 36 and is bisected by the vessel 40.

Turning to FIG. 3A, the vessel is shown in a state in which it has not recently been shaken, thereby allowing sufficient time for the particles 44 to fall to the base of the vessel 42. Accordingly, a large proportion of the light from the LED 35 will pass along direct path C to be detected by the pin photodiode 36 upon interrogation by the controller 22. The controller manipulates the signal from the optical sensor 34 by software or firmware algorithm to determine that the intensity of the light detected by the pin photodiode 36 indicates that an adequate shake has not occurred sufficiently close to the time of dose release.

Turning to FIG. 3B, the vessel is shown in a state in which it has been recently shaken, thereby causing the particles 44 to be dispersed throughout the vessel 42. Accordingly, a large proportion of the light from the LED 35 will be dispersed by the particles 44 along multiple light paths B causing a reduction in the intensity of the light detected by the pin photodiode 36 upon interrogation by the controller 22. The controller manipulates the signal from the optical sensor 34 by software or firmware algorithm to determine that the intensity of the light detected by the pin photodiode 36 indicates an adequate shake has occurred sufficiently close to the time of dose release. A time-stamped electronic record is made of the dosing event in the controller 22, along with an electronic record of the shaking.

It will be appreciated that the movement of particles 42 in the fluid 44 is governed by Stokes Law. Accordingly, the relevant parameters of particles and fluid can be selected to tune the sensitivity of the sensor 30″ to indicate a shake event within a predetermined time range, for example 10 seconds, prior to the user waking the inhaler 10.

In an alternative embodiment of sensor 30″, two solid materials of different types and colours can be used. For example, sedimenting flakes of a blue material and creaming flakes of a red material could be employed, with a suspended purple mixture indicating adequate recent shaking.

In a yet further embodiment of sensor 30″, the suspension is a small, separate portion of the actual medicament formulation being dispensed by the inhaler, in order that its suspension and settling characteristics closely match (other than in its container dimensions and its head space) that of the actual drug formulation being administered.

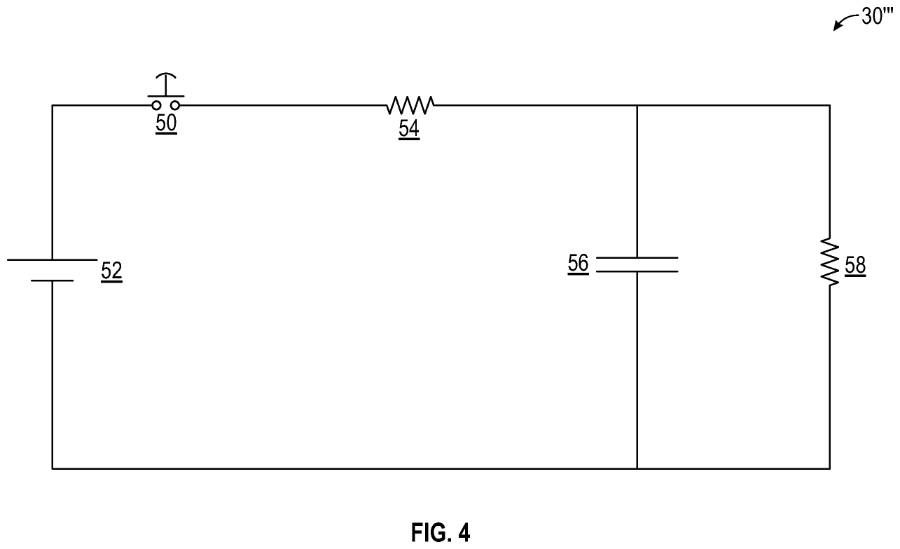

Turning now to FIG. 4, a third embodiment of sensor 30 is shown as an electronic charge capture circuit 30′″. The electronic charge capture circuit 30′″ is used to store energy indicative of recent shaking.

The electronic charge capture circuit 30′″ has a vibration sensitive switch 50 to close the circuit 30′″ in response to the inhaler 10 being shaken by the user. The circuit takes power from a battery 52 which is separate to the main inhaler battery 24 and which causes current to flow through a resistor 54 to charge a capacitor 56. The switch 50 is spring-loaded, so that it is only closed during active shaking motions. Adequate agitation thus causes the switch to repeatedly close, thereby repeatedly briefly closing the circuit 30′″ and charging the capacitor 56 in multiple steps. A second resistor 58 acts to short-circuit the capacitor 56, thus causing it to discharge its voltage whenever it is charged. The resistance of the second resistor 58 is such that the discharge of the capacitor 56 is relatively slow, leading to a gradual decay of the charge in the capacitor 56 and hence the voltage across it. The voltage across the capacitor constitutes a signal indicative of any recent shake event.

In use, the controller 22 reads the voltage across the capacitor 56 when the inhaler is awoken by the user by the opening the mouthpiece cover 18. The controller 22 interrogates the voltage decay signal across the capacitor 56 by appropriate algorithm to determine the adequacy of any shake event prior to wake up.

By way of example, the electronic charge capture circuit 30′″ can be formed as follows:
Battery 52—3V CR1220—RS (RadioSpares) 513-2837.
Capacitor 56—Electrolytic 1 uF.
Resistor 54—330 KOhm.
Resistor 58—10 MegOhm.
Vibration switch 50—(2 to 4.9 g)—RS 455-3665.

A capacitor charges to approximately 63% of the applied supply voltage after one time constant or time period, τ, and to over 99% of the applied voltage over 5τ, where the time constant τ=RC. In this circuit, the time constant, R2×C1, was thus chosen to be 3.3×10⁵ Ohms×10⁻⁶ Farads=0.33 seconds. To fully charge the capacitor to the supply voltage of approximately 3 Volts from the battery thus required approximately 1.65 seconds of closure of the vibration switch.

Table 1 below shows the voltage measured across the capacitor 56 as a function of time, during a series of 7 shakes followed by 3 more shakes over 6 seconds.

TABLE 1

| After shake # (over 2 seconds) | Approx. voltage (from scope) |
|---|---|
| 1 | 0.4 |
| 2 | 0.7 |
| 3 | 0.9 |
| 4 | 1.2 |
| 5 | 1.4 |
| 6 | 1.5 |
| 7 | 1.6 |

One shake comprised an approximately 0.2 metres downwards hand shake followed by an upwards return over a period of approximately 0.3 seconds. The shaking switch used required approximately 2 g (˜19.6 ms⁻²) of acceleration to operate it.

1. An inhaler having an air inlet, a medicament source, and an outlet for delivering an air and medicament admixture to a user, the inhaler including
a primary power supply for powering the controller and
a sensor for detecting a recent shake event,
wherein, the sensor is independent of the primary power source and, in use, the sensor provides the controller with a signal indicative of any recent shake event upon the powering up of the controller prior to use of the inhaler.

2. An inhaler according to claim 1, wherein any recent shake event is a shake event occurring in the range of up to 5 seconds, preferably up to 8 seconds and most preferably up to 10 seconds, prior to the powering up of the controller.

3. An inhaler according to claim 1, wherein the controller interrogates the sensor upon powering up to determine the presence or absence of a signal indicative of a recent shake event.

4. An inhaler according to claim 3, wherein the controller indicates to the user that the inhaler must be shaken in the event of an absence of a signal indicative of a recent shake event.

5. An inhaler according to claim 3, wherein the controller processes the signal indicative of a recent shake event to determine whether the shake event was sufficient to mix the medicament admixture for subsequent inhalation by the user.

6. An inhaler according to claim 5, wherein the controller determines that the recent shake event was sufficient to mix the medicament admixture for subsequent inhalation by the user and advises the user accordingly.

7. An inhaler according to claim 5, wherein the controller determines that the recent shake event was insufficient to mix the medicament admixture and the controller indicates to the user that the inhaler must be shaken prior to use.

8. An inhaler according to claim 1, wherein the sensor is a mechanical sensor which exhibits residual movement initiated by the shake event.

9. An inhaler according to claim 8, wherein the residual movement of the mechanical sensor is the signal indicative of any recent shake event.

10. An inhaler according to claim 9, wherein the inhaler includes an optical sensor powered by the primary power source and the controller interrogates the mechanical sensor using the optical sensor upon powering up of the controller to determine whether any shake event was sufficient to mix the medicament admixture.

11. An inhaler according to claim 8, wherein the mechanical sensor comprises an oscillating mass or a rotating mass.

12. An inhaler according to claim 1, wherein the sensor is a fluid suspension containing a plurality of suspended particles which disperse during a shake event and settle out of suspension over a period of time after the shake event.

13. An inhaler according to claim 12, wherein the extent of dispersion of the suspended particles is the signal indicative of any recent shake event.

14. An inhaler according to claim 13, wherein the inhaler includes a photoelectric sensor powered by the primary power source and the controller interrogates the fluid suspension sensor using the photoelectric sensor upon powering up of the controller to measure the opacity of the fluid suspension in order to determine whether any recent shake event was sufficient to mix the medicament admixture.

15. An inhaler according to claim 1, wherein the sensor is an electronic charge capture circuit which captures a charge indicative of any recent shake event.

16. An inhaler according to claim 15, wherein the circuit comprises:
a vibration sensitive switch;
a battery independent of the primary power supply;
a capacitor for storing the charge;
a first resistor arranged in series; and
a second resistor arranged in parallel with the capacitor.

17. An inhaler according to claim 16, wherein the voltage across the capacitor is the signal indicative of any recent shake event

18. An inhaler according to claim 16 wherein the controller measures the voltage across the capacitor upon powering up of the controller in order to determine whether any recent shake event was sufficient to mix the medicament admixture.

19. An inhaler according to claim 1, wherein the powering up of the controller is initiated by the user pressing a power button on the inhaler or opening a mouthpiece cover of the inhaler.

20. A method of detecting a recent shake event in a medicinal inhaler, comprising:
providing an inhaler having an air inlet, a medicament source, and an outlet for delivering an air and medicament admixture to a user, a primary power supply for powering the controller, and a sensor for detecting a recent shake event, wherein the sensor is independent of the primary power source;
powering up the controller by switching on the inhaler;
interrogating the sensor with the controller to determine the presence or absence of a signal indicative of any recent shake event; and
indicating to the user with controller whether the inhaler must be shaken prior to use.
